(19) United States
(12) Patent Application Publication
(10) Pub. No.: US 2025/0116740 A1
(43) Pub. Date: Apr. 10, 2025

(54) SYSTEM AND METHOD FOR DETECTING THE TRANSPORT OF SUBSTANCES ACROSS A BIOLOGICAL BARRIER

(71) Applicant: Jinghua Wang, Mason, OH (US)

(72) Inventor: Jinghua Wang, Mason, OH (US)

(73) Assignee: Jinghua Wang, Mason, OH (US)

(21) Appl. No.: 18/164,621

(22) PCT Filed: Jul. 13, 2021
(86) PCT No.: PCT/US2021/070869

§ 371 Date: Feb. 6, 2023

Related U.S. Application Data
(60) Provisional application No. 63/072,290, filed on Aug. 31, 2020.

(51) Int. Cl. G01R 33/563; A61B 5/055; G01R 33/56

(52) U.S. Cl. CPC G01R 33/56341; A61B 5/055; G01R 33/5601

(57) ABSTRACT

The present invention relates to a method and system for identifying the movement of a substance across a biological barrier using diffusion magnetic resonance imaging (dMRI). The method involves administering an exogenous substance to a subject undergoing imaging, and then obtaining one or more images of targeted regions post-administration using dMRI. The images are analyzed to characterize changes in diffusivity, permeability, or diffusion tensor parameters, enabling the determination of the transport of the exogenous substance through the biological barrier.

### CROSS-REFERENCE TO RELATED APPLICATION

This application claims priority to the benefit of the filing date of U.S. Provisional Application No. 63/072,290, entitled “System and method for detecting the transport of substances across blood-brain barrier” filed on Aug. 31, 2020, which is hereby incorporated by reference in its entirety.

### BACKGROUND

### Field of the Invention

The present invention relates to systems and methods for detecting the transport of a substance across a biological barrier through diffusion magnetic resonance imaging after the administration of the substance.

### Description of the Related Art

Magnetic resonance imaging (MRI) is one of the most important modern medical imaging modalities. It has far less risk of side effects than most other imaging modalities, such as radioscopy with x-rays or computed tomography, because patients and medical personnel are not subjected to ionizing radiation exposure in the procedure. The use of MRI has grown very fast. Every year, more than 30 million MRI scans are performed in the United States; more than 60 million MRI scans are performed worldwide. Doctors often recommend MRI for the diagnosis of various diseases, such as tumors, strokes, heart problems, and spine diseases. A high-quality scan is important for maximizing diagnostic sensitivity and accuracy. Generally, high quality images are characterized by high signal-to-noise ratio (SNR), high contrast between normal and pathological tissues, low levels of artifacts, and appropriate spatial-temporal resolution.

In order to obtain a detectable MR signal, the object/subject examined is positioned in a homogeneous static magnetic field, so that the object's nuclear spins generate net magnetization oriented along the static magnetic field. The net magnetization is rotated away from the static magnetic field using a radio frequency (RF) excitation field with the same frequency as the Larmor frequency of the nucleus. The angle of rotation is determined by the field strength of the RF excitation pulse and its duration. In the end of the RF excitation pulse, the nuclei, in relaxing to their normal spin conditions, generate a decaying signal (the “MR signal”) at the same radio frequency as the RF excitation. The MR signal is picked up by a receive coil, amplified and processed. The acquired measurements, which are collected in the spatial frequency domain, are digitized and stored as complex numerical values in a “k-space” matrix. An associated MR image can be reconstructed from the k-space data, for example, by an inverse 2D or 3D fast Fourier transformation (FFT) from the raw k-space data.

Biological barrier is defined as a living organism that helps protect the body from pathogens, such as a blood-brain barrier (BBB), cell membrane, nuclear membrane, skin, mucosal membranes, blood-cerebrospinal fluid (CSF) barrier, and blood-tumor lesion barrier. These barriers are designed by nature to keep foreign material out and only allow small molecules with specific characteristics to cross. For example, BBB controls the transport of nutrients and energy metabolites into the brain and wash-outed waste substances circulating in the bloodstream. BBB disruption associates with numerous brain diseases, and neurovascular dysfunction as well as neurodegeneration. It has been used as a significant marker for a wide variety of diseases such as brain tumors, Parkinson's disease, traumatic brain injury, vascular cognitive impairment, multiple sclerosis, stroke, chronic vascular disease, and in disorders with a primary neurodegenerative component such as Alzheimer's disease and dementia. BBB disruption is also a common pathological finding in many psychiatric disorders including schizophrenia, autism spectrum disorder (ASD) and mood disorders. The severity of various diseases is proportional to the degree of BBB disruption. The highly organized BBB structure is also the major obstacle preventing lifesaving drugs from reaching the brain to effectively treat cancer, neurodegeneration, and other diseases of the central nervous system. In a number of brain diseases, the BBB can also break down locally, causing neurotoxic substances, blood cells, and pathogens to leak into the brain and wreak irreparable havoc.

Generally, transport of substances to the brain can be categorized into two main areas: bypassing the BBB and crossing the BBB. There remains an unmet need to develop a novel method for estimating transport of substances across BBB, such as imaging contrast agents and drugs. Measuring BBB permeability in humans is not straightforward. The cerebrospinal fluid/serum albumin ratio is a common and well-established method to assess BBB permeability, but it is invasive and there are concerns that it does not reliably reflect BBB permeability. Medical imaging after an intravenous injection of contrast agent is an attractive technique to measure BBB permeability. BBB disruption of pathological tissue is estimated by measuring a tracer or contrast agent, which does not cross BBB under normal physiological conditions. The contrast agents used for estimating BBB are either exogenous or endogenous. Many image modalities are available to quantitatively measure BBB disruption, including computed tomography (CT), single photo-emitting computed tomography (SPECT) and MRI. The use of SPECT is limited because of the low spatial resolution, less sensitive for small BTB lesions and ionizing radiation. The advantages of using dynamic contrast enhanced-CT (DCE-CT) include short exam time and better availability than MRI in clinical settings. However, its applications are limited by some disadvantages, including ionizing radiation, bad soft tissue contrast and an increasing risk for adverse reactions caused by iodinated contrast agent. Currently, dynamic contrast agent MRI, a T₁-weighted acquisition dynamic contrast-enhanced magnetic resonance imagining (DCE-MRI), and a T₂*-weighted acquisition dynamic susceptibility contrast (DSC-MRI), can be used to measure BBB permeability which has some advantages, including no ionizing radiation, and good soft tissue contrast. Although DCE-MRI has been shown to be a robust research tool, it does not become part of standard clinical practice yet. This is partly due to some of the limitations of DCE-MRI, including expensive procedure, multicompartment physiologic models, complex data analysis methods and long exam time. Dynamic susceptibility contrast magnetic resonance imaging (DSC-MRI) is also used to estimate BBB disruption. Because the bolus transit time for DSC-MRI imaging is so short, a fast acquisition technique provides the necessary temporal resolution to adequately characterize the transient drop in signal intensity. DSC-MRI is also limited by susceptibility artifacts and contrast leakage. Additionally, permeability of BBB in neurodegenerative diseases is about several percent of BBB permeability in the brain tumor, particularly at the early stage of the diseases. Currently, dynamic contrast MRI techniques are one of the most sensitive methods to clinically detect early changes in BBB permeability. But the reliable measurement of low-level BBB permeability remains a difficult problem in DCE-MRI. In order to obtain precise results about BBB disruption, the longer scan time is required. It also implies that the accumulation of the BBB disruption influenced the detection sensitivity. Over past decade, numerous contrast agents used for detecting BBB permeability are disclosed as follows:

U.S. Patent Application Publication No. 2012/0179028A1 to Peter Caravan et al. discloses a method to measure a permeability of a subject's blood-brain barrier to water by the two T₁ maps acquired at the different time frames before and after administration of a contrast agent.

U.S. Pat. No. 9,194,867 B2 to Aristo Vojdani discloses methods, assays, and apparatus for testing of antigens (such as blood, saliva or other bodily fluid) associated with intestinal and/or BBB permeability.

U.S. Patent Application Publication No. 2015/0265210 A1 to David Israeli et al. discloses a method of analyzing a blood-brain barrier of a subject having a detectable dose of an MRI contrast agent by comparing two or more of the magnetic resonance images so as to determine variations in concentration of the contrast agent in the organ.

U.S. Pat. No. 9,046,589 B2 to Kjell-Inge Gjesdal et al. discloses methods, apparatus, and computer based systems for identifying benign and malignant tumors in tissues such as soft tissues and particularly breast tissue using DCE-MRI and dynamic susceptibility contrast-enhanced magnetic resonance imagining (DSC-MRI) of the tumors. This success for both methods is due to the methods' ability to identify physiological differences in cancer tissue through the quantifying of the contrast agent in the tissue over time.

U S. Patent Application Publication No. 2014/0086827 A1 to Damir Janigro et al. discloses a method of assessing blood brain barrier permeability through S 100BB homodimer.

International Publication No. WO 2014/205338A3 and U.S. Patent Application Publication No. 2016/0120893A1 to Chenghua Gu and Ayal Ben-Zvi disclose a method to modulate the permeability of the blood-brain barrier for therapeutic purposes.

U.S. Pat. No. 9,291,692 B2 to Feng-Yi Yang et al. discloses a method of assessing the blood-brain barrier recovery curve using a focused ultrasound DCE-MRI technique.

International Publication No. WO 2016/042554A1 and U.S. Patent Application Publication No. 2017/0247429A1 to Itzik Cooper et al. discloses a method to develop novel BBB penetrating agents for the treatment of brain diseases and disorders.

International Publication No. WO 2015/138974A1 and U.S. Pat. No. 9,913,899 B2 to Lawrence M. Kauvar and Damir Janigro discloses a method to test the blood of a patient for total S-100B or for S-100BB as a marker of blood brain barrier integrity to reduce neuronal damage caused by a cerebral ischemic event in a human patient.

International Publication No. WO 2017/021951A1 to Alon Friedman and Yehuda Vazana discloses a method for reducing the permeability of the blood-brain-barrier in a patient by administration of a composition which includes N-methyl-d-aspartate receptor antagonists.

International Publication No. WO 2017/048778A1 and U.S. Patent Application Publication No. 2018/0256756A1 to Richard D. Kopke and Rheal A. Towner disclose a method to transport a therapeutic or diagnostic agent across a blood-brain barrier or a blood-cochlear barrier or a blood-cerebrospinal fluid barrier of a subject by administration of 2,4-disulfonyl a-phenyl tertiary butyl nitrone.

International Publication No. WO 2017/049411A1 and U.S. Patent Application Publication No. 2018/0250361A1 to Philippe Patrick Monnier et al. disclose a method to modulate the permeability of the blood brain barrier for the treatment of diseases, and promote re-myelination as well as prevent de-myelination.

U.S. Pat. No. 10,076,263B2 to Richard Leigh and Peter B. Barker discloses a method to estimate blood brain permeability imaging using dynamic susceptibility contrast magnetic resonance imaging.

The paper “Non-contrast MR imaging of blood-brain barrier permeability to water” in Magnetic Resonance in Medicine. 2018; 80:1507-1520 to Zixuan Lin et al. proposed a method using conventional arterial-spin-labeling with very long post-labeling delays for quantifying blood-brain barrier permeability.

The paper “Mapping water exchange across the blood-brain barrier using 3D diffusion-prepared arterial spin labeled perfusion MRI” Magnetic Resonance in Medicine. 2019; 81:3065-3079 to. Xingfeng Shao et al. proposed a new pseudo-continuous arterial spin labeling sequence with a diffusion preparation and 3 dimensional gradient and spin echo readout for quantifying blood-brain barrier permeability.

The paper “Dynamic glucose-enhanced (DGE) MRI: translation to human scanning and first results in glioma patients” in Tomography. 2015; 1: 105-114 to Xing Xu et al. disclosed a new chemical exchange saturation transfer method to identify BBB disruption in glioma patients.

To date, most contrast agent MRI methods for determining permeability focused on the property of T₁ and T₂* changes after the administration of the T₁ and T₂* contrast agents. Additionally, monitoring drug delivery is deemed highly challenging, if not unfeasible.

This background information is provided for the purpose of making known information believed by the applicant to be of possible relevance to the present invention. No admission is necessarily intended, nor should be construed, that any of the preceding information constitutes prior art against the present invention.

### SUMMARY

The present methods and systems use the diffusivity of substances after the administration of the substances to determine the permeability and/or the enhanced image contrasts by diffusion magnetic resonance imaging (dMRI). The dMRI according to the present disclosure can characterize the transport of many drugs or foods across a biological barrier entering human tissues.

This disclosure describes method and system to detect the transport of a substance across a biological barrier after the administration of the substance using diffusion magnetic resonance imaging (dMRI) sequence herein. It should be understood that this disclosure contemplates using dMRI sequence imaging, which is provided as an example only, with the techniques described herein. Alternatively or additionally, the transport of a substance across the biological barrier after the administration of the substance, is detected by intra-voxel incoherent motion (IVIM) or diffusion tensor imaging.

In one embodiment, a method for detecting the transport of an exogenous substance across a biological barrier such as a blood-brain barrier using a diffusion magnetic resonance imaging sequence is provided. The method includes administrating an exogenous substance to target regions including human tissues or lesions; acquiring one or more images of target regions after the administration of the exogenous substance by the diffusion magnetic resonance imaging sequence; processing the one or more images to identify diffusivity, permeability, or diffusion tensor parameters of the target regions; and characterizing the transport of the exogenous substance across the biological barrier based on the identified diffusivity, permeability, or diffusion tensor parameters of the target regions.

In another embodiment, a system for detecting the transport of an exogenous substance across a biological barrier using a dMRI sequence is provided. The system includes a coil configured to apply a dMRI sequence, a receiver configured to acquire one or more images of target regions after the administration of the exogenous substance by the dMRI sequence, and a processor configured to process the one or more images to identify diffusivity, permeability, or diffusion tensor parameters of the target regions, and characterize the transport of the exogenous substance across the biological barrier based on the identified diffusivity, permeability, or diffusion tensor parameters of the target regions.

In some implementations, the detection of a substance across a blood-brain barrier (BBB) after the administration of the substance can be implemented for the detection and treatment assessment of various diseases, including tumor, multiple sclerosis, Parkinson's disease, vascular cognitive impairment, chronic vascular disease but not limited to, inflammation disease, infection disease, stroke, traumatic nerve injury, vascular disease, Alzheimer's disease, dementia, schizophrenia, autism spectrum disorder and mood disorders.

Optionally, said substance is a drug used for treatment. Alternatively or additionally, the detection of a drug across BBB after the administration of the drug can be further implemented to monitor a drug delivery for both the development and clinical application of the drug.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a diagram illustrating an example MRI system.

FIG. 1A is an example computing device.

FIG. 2 is a flowchart illustrating example operations for detecting the transport of a substance across a biological barrier after the administration of the substance using a diffusion magnetic resonance imaging sequence.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not.

The term “substance” as used herein any natural or artificial material with a definite molecular structure. It can be one or more of compound, polymer, nanoparticle, composition, biological entity or their variants that can pass a biological barrier. In one embodiment, the substance can be a contrast agent for magnetic resonance imaging (MRI), computer tomography (CT) imaging, ultra-sound imaging, positron emission tomography imaging (PET), and other medical imaging. MRI contrast agents include one or more of gadopentate dimeglumine, Gadobutrol, Gadoterate meglumine, Gadoteridol injection, Ferric ammonium citrate, Manganese Chloride, Ferristene, Ferumoxides, and their variants. CT contrast agents include one or more of Ethiodized oil, Ioversol, Iohexol, Iopromide, Iodixanol, Ioxaglate, Iothalamate, Iomeprol, Ioxilan, Ioxaglate, and Iopamidol. Ultra-sound contrast agents include one or more of Albunex, Levovist, Optison and Sonazoid, and Lumason.

The term “biological barrier” as used as herein refers to a selectively permeable barrier which separates cell from the external environment or creates intracellular compartments. The biological barrier includes one or more of cell membrane, nuclear membrane, blood-tissue barrier (BTB), skin, mucosal membranes, blood-cerebrospinal fluid (CSF) barrier, blood-retinal barrier, blood-testis barrier, blood-tumor lesion barrier, and blood brain barrier.

The term “blood brain barrier (BBB)” as used herein is a highly selective border that separates the circulating blood from the brain. The blood-brain barrier is composed of endothelial cells of the capillary wall, astrocyte end-feet ensheathing the capillary, and pericytes embedded in the capillary basement membrane. BBB greatly restrict exchange of substances between capillaries and brain tissues. Though the present system and method herein focus on characterizing the transport of substance across BBB, they can be extended to other biological barriers.

The term “exogenous contrast agent” or “exogenous substance” as used interchangeably herein is a substance used to increase the contrast or improve more information of structures or fluids within the body in medical imaging. The exogenous contrast agent may be used to diagnose disease as well as monitor treatment effects. The exogenous contrast agent may be administered by oral or intravenous administration. For example, MRI contrast agents are used to improve the visibility of internal body structures. The most commonly used compounds for contrast enhancements are gadolinium-based compounds which shorten the relaxation times following oral or intravenous administration. The disadvantage of exogenous contrast agents is that there are side effects associated with the administration of the contrast agents. For example, the injection of Gd-based contrast agent in MRI has potential side effects of nephrogenic systemic fibrosis (NSF), deposition of Gd molecules, and potential neurotoxicity.

The term “endogenous contrast agent” as used herein depends on the intrinsic ability of human organs to increase the contrast or improve more information of structures or fluids within the body in medical imaging. For example, arterial spin labelling MRI uses magnetically labeled arterial blood water protons as an endogenous contrast agent to measure tissue perfusion. The endogenous contrast agent method is very promising in clinical screening and management because its injection does not pose potential risk to patients.

The term “administration” and its variant as used herein refers to introducing a substance into a subject. In some embodiments, a route of administration is oral administration or intravenous administration. However, any route of administration, such as topical, subcutaneous, peritoneal, intraarterial, inhalation, vaginal, rectal, nasal, introduction into the cerebrospinal fluid, or instillation into body compartments can be used.

The terms “therapy” and “treatment” as used interchangeably herein, refer to an intervention performed with the intention of improving a subject's status.

The terms “detection” and “diagnosis” as used interchangeably herein, refer to identify the abnormal tissue or lesion.

The term “drug” as used herein is defined as a medicament or medicine which is used for the therapeutic treatment of a medical condition or disease. The drug may be used in combination with another drug or type of therapy.

The term “variant” as used herein refers to a substance that differs from a reference substance, but retains essential properties. A variant of a substance can be modified by substitutions, additions, and/or deletions of the chemical elements. For example, new contrast agents may be variants of existing substances that can pass biological barriers, but their diffusivity is modulated. The variant may be naturally occurring, or it may be a variant that is not known to occur naturally.

### MRI System Overview

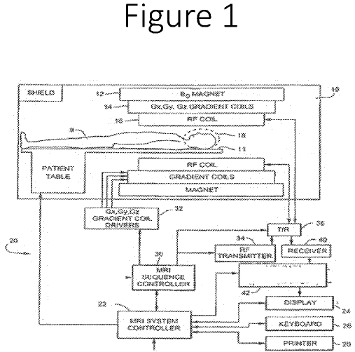

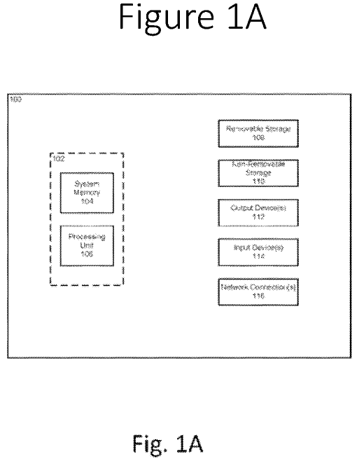

FIG. 1 depicts an MRI system 10, according to one or more embodiments described and shown herewith. In embodiments, the MRI system 10 shown in FIG. 1 includes a patient table 11, a static magnetic field generating unit 12, a gradient magnetic field generating unit 14 for generating respective magnetic fields in proximity to a target area 18 of an object 9, a transmitting and receiving unit 16, and a computing device 100. The patient table 11, the static magnetic field generating unit 12, the gradient magnetic field generating unit 14, and the transmitting and receiving unit 16 are placed within MRI RF shielding area 2 where noise of radio frequency is prevented from entering.

The static magnetic field generating unit 12 includes a main magnet configured to generate a strong static magnetic field in proximity to the target area 18 of the object 9. The static magnetic field generating unit 12 may be arranged to surround the target area 18 of the object 9. For example, the static magnetic field generating unit 12 may be a cylindrical-shaped unit. The gradient magnetic field generating unit 14 includes gradient magnetic field coils for generating gradient magnetic fields in an x-axis direction, a y-axis direction, and a z-axis direction, which are orthogonal to each other. The gradient magnetic field generating unit 14 may be arranged to surround the target area 18 of the object 9. For example, the gradient magnetic field generating unit 14 may be a cylindrical-shaped unit.

In embodiments, the transmitting and receiving unit 16 may include a transmission coil and a receiving coil. The transmission coil irradiates RF pulses to the object 9 and the receiving coil receives MR signals generated by the object 9. In some embodiments, the transmitting and receiving unit 16 may include a transceiver coil having the functions of both the transmission coil and the receiving coil. The receiving coil may be composed of, for example, a so-called array coil in which, for example, a plurality of coil elements are disposed to detect the MR signals generated by the object 9. An RF transmitter 34 may control the transmission coil of the transmitting and receiving unit 16 to irradiate RF pulses. A receiver 40 may receive MR signals generated by the object 9 from the receiving coil of the transmission and receiving unit 16. The RF transmitter 34 and the receiver 40 may communicate with the transmitting and receiving unit 16 through a transmitter/receiver interface 36.

In embodiments, the MRI system 10 employs diffusion magnetic resonance imaging (dMRI). The MRI system 10 uses specific MRI sequences and software that generates images from the resulting data that uses the diffusion of water molecules or other substances to generate contrast in MR images. Molecular diffusion in tissues reflects interactions among different molecules, including water molecule, contrast agent molecule, drug molecule and other substance molecules.

In embodiments, the MRI system 10 includes the computing device 100. The computing device 100 includes a MRI system controller 22. The MRI system controller 22 may control the operations of the gradient coil drivers 32 that activate the gradient coils of the gradient magnetic field generating unit 14. The MRI system controller 22 may also control the operations of the RF transmitter 34 that activates the RF coil of the static magnetic field generating unit 12. The computing device 100 may receive MR signals from the receiving coil of the transmission and receiving unit 16 and reconstruct an MRI image based on the received MR signals. The details of the computing device 100 will be further described with reference to FIG. 1A below.

In embodiment, the computing device 100 may be operably coupled to other components of the MRI system 10, for example, using by any medium that facilitates data exchange between the components of the MRI system 10 and the computing device 100 including, but not limited to, wired, wireless and optical links. For example, the computing device 100 may convert the MR signals received from the transmitting and receiving unit 16 into k-space data. The computing device 100 may generate MR image data from the k-space data with image reconstruction processing. In some embodiments, the techniques for improving image quality with optimal variable flip angles may optionally be implemented using the MRI system 10.

### Example Computing Device

FIG. 1A depicts a computing device 100 according to one or more embodiments shown and described herein. It should be appreciated that the logical operations described herein with respect to the various figures may be implemented (1) as a sequence of computer implemented acts or program modules (i.e., software) running on a computing device (e.g., the computing device described in FIG. 1A), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

It should be understood that the computing device 100 is only one example of a suitable computing environment upon which embodiments of the invention may be implemented. Optionally, the computing device 100 may be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In embodiments, the computing device 100 includes a controller 102 that includes one or more processing units 106 and one or more system memory modules 104. The controller 102 may be the same controller as the MRI system controller 22 in FIG. 1. In other embodiments, the controller 102 may be a separate controller from the MRI system controller 22 in FIG. 1. Depending on the exact configuration and type of computing device, the one or more memory modules 104 may be volatile (such as random access memory (RAM)), non-volatile (such as read-only memory (ROM), flash memory, etc.), or some combination of the two. The one or more processing units 106 may be a standard programmable processor that performs arithmetic and logic operations necessary for operation of the computing device 100.

In embodiments, the computing device 100 includes communication path 120 that provides signal interconnectivity between various components of the computing device 100. Accordingly, the communication path 120 may communicatively couple any number of processing units 106 with one another, and allow the components coupled to the communication path 120 to operate in a distributed computing environment. Specifically, each of the components may operate as a node that may send and/or receive data. As used herein, the term “communicatively coupled” means that coupled components are capable of exchanging data signals with one another such as, for example, electrical signals via conductive medium, electromagnetic signals via air, optical signals via optical waveguides, and the like.

Accordingly, the communication path 120 may be formed from any medium that is capable of transmitting a signal such as, for example, conductive wires, conductive traces, optical waveguides, or the like. In some embodiments, the communication path 120 may facilitate the transmission of wireless signals, such as Wi-Fi, Bluetooth, Near Field Communication (NFC) and the like. Moreover, the communication path 120 may be formed from a combination of mediums capable of transmitting signals. In one embodiment, the communication path 120 comprises a combination of conductive traces, conductive wires, connectors, and buses that cooperate to permit the transmission of electrical data signals to components such as processors, memories, sensors, input devices, output devices, and communication devices. Accordingly, the communication path 120 may comprise a vehicle bus, such as for example a LIN bus, a CAN bus, a VAN bus, and the like. Additionally, it is noted that the term “signal” means a waveform (e.g., electrical, optical, magnetic, mechanical or electromagnetic), such as DC, AC, sinusoidal-wave, triangular-wave, square-wave, vibration, and the like, capable of traveling through a medium.

The one or more processing units 106 may be configured to execute program code encoded in tangible, computer-readable media. Tangible, computer-readable media refers to any media that is capable of providing data that causes the computing device 100 (i.e., a machine) to operate in a particular fashion. Various computer-readable media may be utilized to provide instructions to the one or more processing units 106 for execution. Example tangible, computer-readable media may include, but is not limited to, volatile media, non-volatile media, removable media and non-removable media implemented in any method or technology for storage of information such as computer readable instructions, data structures, program modules or other data. One or more system memory modules 104, a removable storage 108, and a non-removable storage 110 are all examples of tangible, computer storage media. Tangible, computer-readable recording media may include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In embodiments, the one or more processing units 106 may execute program code stored in the one or more system memory modules 104. For example, a bus may carry data to the one or more system memory modules 104, from which the one or more processing units 106 receive and execute instructions. The data received by the one or more system memory modules 104 may be optionally stored on the removable storage 108 or the non-removable storage 110 before or after execution by the processing unit 106.

In embodiments, the computing device 100 may include additional storage such as removable storage 108 and non-removable storage 110 including, but not limited to, magnetic or optical disks or tapes.

The computing device 100 may also have input device(s) 114 such as a keyboard, mouse, touch screen, etc. The input device may be manipulated by an operator to input signals to the MRI apparatus to set the imaging method group, the performing order, the imaging condition, and the like. The computing device 100 may also have output device(s) 112 such as a display, speakers, printer, etc. The output device 112 may output image data such as local image data, diagnosis image data using display, printer and other displayer. The additional devices may be connected to the bus in order to facilitate communication of data among the components of the computing device 100.

Computing device 100 may also contain network connection(s) 116 that allow the device to communicate with other devices. The network connection(s) 116 may be any device capable of transmitting and/or receiving data via a wireless network. Accordingly, the network connection(s) 116 may include a communication transceiver for sending and/or receiving data according to any wireless communication standard. For example, the network connection(s) 116 may include a chipset (e.g., antenna, processors, machine readable instructions, etc.) to communicate over wireless computer networks such as, for example, wireless fidelity (Wi-Fi), WiMax, Bluetooth, IrDA, Wireless USB, Z-Wave, ZigBee, or the like.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

In some embodiments, the computing device 100 may include a workflow setting unit, an imaging operation determining unit, and an image reconstruction unit. The workflow setting unit may be a program module stored in the system memory modules 104. The workflow setting unit sets a first workflow relating to the MRI examination by estimating an imaging time of each of the imaging methods in the performing order initially set by a scan plan. Further, the workflow setting unit sets a second workflow relating to the MRI examination by estimating a shortest performing order, by which an examination time necessary to sequentially perform a plurality of imaging methods constituting the imaging method group set by the input unit is minimized. The imaging operation determining unit determines whether an imaging operation during a main imaging is implemented according to the workflow. In embodiments, the workflow setting unit and/or the imaging operation unit may be implemented using hardware, software, and or a combination thereof.

The image reconstruction unit may include an MR signal storage unit, a signal processing unit, and an image data storage unit. The MR signal storage unit (e.g., memory) stores the MR signals, which are collected by the receiver unit of the transmitting and receiving unit 16. The signal processing unit has an image reconstruction processing unit and an image processing unit. The image reconstruction processing unit generates image data from the MR signal storage unit by image reconstruction processing, for example, performed by a Fourier transformation such as 2D FFT. When the MR signals to a three-dimensional region are collected, the image reconstruction processing unit of the signal processing unit generates volume data. Subsequently, the image processing unit generates three-dimensional image data such as volume rendering image data, surface rendering image data and the like or two-dimensional image data, multi planar reconstruction image data, and the like, because predetermined image processing is performed for the volume data generated by the image reconstruction processing unit. Then, the image data described above obtained by the signal processing unit are stored to the respective storage regions of the image data storage unit.

### Method

The blood-brain barrier (BBB) is defined as a selective diffusion barrier at the level of the cerebral microvascular endothelium that is characterized by the lack of fenestrations and the presence of tight junctions (TJs) on endothelial cells. The present system and method herein focus on characterizing the transport of substance across BBB. Furthermore, they can be extended to other biological barriers, including cell membrane, nuclear membrane, blood-tissue barrier (BTB), skin, mucosal membranes, blood-cerebrospinal fluid (CSF) barrier, blood-retinal barrier, blood-testis barrier, blood-tumor lesion barrier, etc.

BBB disruption is linked to various brain seizures, brain injury, neurodegenerative diseases, and psychiatric disorders. The neurodegenerative diseases include multiple sclerosis, Parkinson's disease, Huntington's disease, Pick's disease, Alzheimer's disease, amyotrophic lateral sclerosis, dementia, and stroke, and peripheral neuropathy. They also include brain tumors and major depression, diabetes, and obesity. The psychiatric disorders includes schizophrenia, autism spectrum disorder and mood disorders. The BBB disruption will lead to the increased permeability of some substances, such as water and Gd-based contrast agent. Therefore, detecting the transport of a specific substance across BBB may be a potential biomarker for the diseases associated with BBB disruption.

Moreover, the therapies for many diseases, such as cancer and neurodegenerative diseases, relate with the BBB. The transport of a substance across BBB is selective and creates major hurdles for successful central nervous system (CNS) drug development. For example, molecules like glucose and lipid soluble drugs can rapidly pass BBB into the brain. Although some effective treatments are available, most of those diseases remain undertreated. The major reason for the undertreated diseases is because BBB limited the delivery of many drug types (such as insoluble drugs) that are very difficult to transport into the brain tissue. As a result, only 5% of the more than 7000 small-molecule drugs available can currently treat CNS diseases. Safe and non-invasive opening of the BBB is a significant challenge for the treatment. Therefore, detecting the transport of a drug across BBB may be a unique tool to understand and monitor the drug delivery non-invasively and in real-time.

A lot of magnetic resonance imaging methods, including contrast enhanced MRI, DCE-MRI, DSC-MRI, arterial spin labeling (ASL) MRI, intravoxel incoherent motion (IVIM), can be used to estimate the transport of a substance that pass BBB to enter brain tissues. For example, ASL method magnetically labels the protons of arterial water, which is used as an endogenous contrast agent to measure tissue perfusion of brain tissues.

Diffusion magnetic resonance imaging (dMRI) can implement specific sequences to in vivo and non-invasively characterize random motion of water molecules. Water molecular diffusion associates with tissue microstructures, such as macromolecules, fibers, and membranes. Therefore, changes of water molecule diffusion in diffusivity and anisotropy can reveal tissue microstructure, either normal or in a diseased state. Conventionally, diffusion imaging sequences are used to identify the changes of structure or connectivity that are caused by physio-pathology. The present invention uses the diffusion imaging sequences to identify the changes in diffusivity of targeted tissues that are caused by the transport of the substances across BBB.

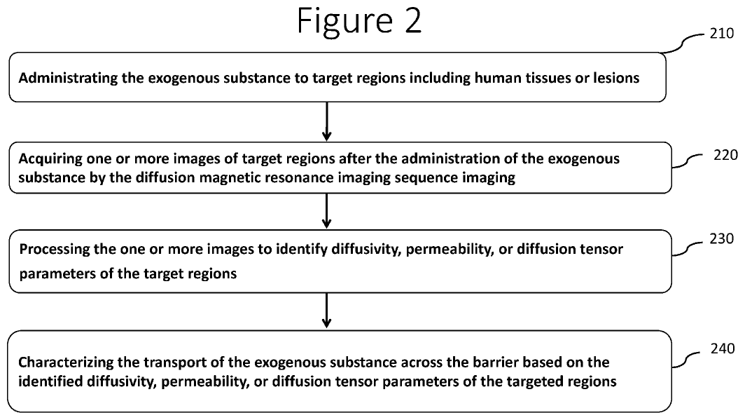

FIG. 2 is a flowchart illustrating example operations for detecting the transport of a substance across a biological barrier after the administration of the substance using a dMRI sequence. The detection of the transport of the substance across the barrier breakdown can include the following steps.

In step 210, an exogenous substance is administered into target regions of a subject. The exogenous substance can be any safe substance which is approved by FDA. The exogenous substance is one or more of existing contrast agents, drugs, nano-particles, food, or nutrient substances and their variants.

In step 220, the MRI system 10 acquires one or more images of target regions after the administration of the exogenous substance by the dMRI sequence.

In step 230, the MRI system 10 processes the one or more images to identify diffusivity, permeability, or diffusion tensor parameters of the target regions.

In step 240, the MRI system 10 characterizes the transport of the exogenous substance across the biological barrier based on the identified diffusivity, permeability, or diffusion tensor parameters of the targeted regions. The contrast between healthy tissue and pathological tissue with biological barrier disruption in the target region can be differentiated by their different diffusivity, permeability, or diffusion tensor parameters based on the image data. In embodiments, the MRI system acquires a first image of the target regions before the administration of the exogenous substance by the diffusion magnetic resonance imaging sequence, acquires a second image or more images of the target regions after the administration of the exogenous substance by the diffusion magnetic resonance imaging sequence, or IVIM, or the diffusion tensor imaging, identifies changes in diffusivity, permeability, or diffusion tensor parameters of the target regions by comparing the first image and the second image of the target regions, and characterizes the transport of the exogenous substance across the biological barrier based on the identified changes in diffusivity, permeability, or diffusion tensor parameters of the target regions.

In embodiments, the MRI system 10 may visually differentiate healthy tissue and physio-pathological changes. For example, only one or more of MRI images may be acquired after the administration of the exogenous, and the MRI system 10 may identify the contrast between healthy tissue and lesion according to one or more of post-contrast images.

In some embodiments, the MRI system 10 may quantify physio-pathological changes by the different diffusivities according to at least two images before and after the administration of the substances, respectively. Image processing may be implemented on the acquired image data to identify very small transport of the substances across the biological barrier using the subtraction of two images before and after the administration of the substances. For example, the computing device 100 may subtract the first image data from the image acquired after the administration of the substances to visually detect the diffusivity or calculate the changes in the real-time diffusivity from several image data that were acquired after administrating the substances. The first image data is acquired before or immediately after the administration of substances.

This disclosure describes methods and systems to detect the transport of the substance cross BBB using dMRI herein. It should be understood that the present method may be available for other image modalities, such as perfusion magnetic resonance imaging, intra-voxel incoherent motion (IVIM), a positron emission tomography (PET) apparatus, a computed tomography (CT) apparatus, and a single positron emission computed tomography (SPECT) apparatus.

Optionally, characterizing the transport of the exogenous substance across the BBB according to the one or more post-contrast image data may be either quantitative or qualitative.

Optionally, the target regions may include at least one of a lesion, a landmark, a texture, or a feature of interest.

### Example Application

The increased BBB transport of a substance is considered as a consequence of ongoing processes like inflammation, atherosclerosis, and lack of vascular-autoregulation, which is linked to various brain seizures, brain injury, neurodegenerative diseases, and psychiatric disorders. Worldwide, neurodegenerative diseases account for more than 20 million patients. Aging greatly increases the risk of neurodegenerative disease while the average age is steadily increasing. In the United States of America, Alzheimer disease (AD) will impact ˜10 million by 2050; Multiple sclerosis (MS) that is the most common demyelinating neurodegenerative disease of CNS, affect an estimated 400,000 people; Stroke that is the fourth leading cause of death in the US, there are more than about 795,000 people with a new or recurrent stroke each year, and 137,000 people dying each year; Parkinson diseases impact approximately 60,000 Americans each year. Additionally, cerebral small vessel disease (CSVD) of the cerebral circulation is a major contributor to hemorrhagic stroke, dementias and other forms of neurological dysfunction, influencing many Americans. A lot of studies indicate that the transport of a substance (input or output of substances) across BBB closely associate with the CNS diseases mentioned above. The present method for detecting the transport of an exogenous substance across BBB using a dMRI sequence provides a new and unique window for better understand of BBB mechanism, diagnosis, treatment assessment, and drug development of various diseases associated with BBB disruption.

Alternatively or additionally, in some implementations, diffusion magnetic resonance imaging sequence is used to characterize the transport of a substance (a contrast agent or a drug) in humans induced by drugs or techniques. For example, highly focused ultrasound or laser-based approaches are used to open a biological barrier so that the therapy drug can cross a biological barrier to deliver to the local lesion. The present method can be used for better understanding of drug delivery during the application of highly focused ultrasound or laser-based approaches.

Specific implementations of the disclosed system and method are useful to illustrate its nature. These examples are non-limiting and are offered as exemplary only.

Alternatively or additionally, in some implementations, the lesion tissue can optionally be at least one of tumor, multiple sclerosis, Parkinson's disease, vascular cognitive impairment, chronic vascular disease but not limited to, inflammation disease, infection disease, stroke, traumatic nerve injury, vascular disease., Alzheimer's disease, dementia, schizophrenia, autism spectrum disorder and mood disorders.

Alternatively or additionally, in some implementations, the contrast agent can optionally be at least one physiologically acceptable paramagnetic substance, superparamagnetic substance, or ferromagnetic substance. Alternatively or additionally, in some implementations, the contrast agent can optionally be at least one of a magnetic small-molecule-based compound, a magnetic large-molecule-based compound, or a magnetic nanoparticle-based compound. Optionally, the contrast agent is administered by injection or orally.

Alternatively or additionally, the contrast agent is endogenous contrast agent which has lower toxicity than drugs and exogenous contrast media, for example, de-oxygenhemoglobin in blood.

Currently, clinical molecular imaging focuses on PET and SPECT imaging. Alternatively or additionally, in some implementations, the present method can combine with specific contrast agents for MRI molecular imaging.

Alternatively or additionally, qualitative differentiating healthy tissue and pathological tissue with blood-tissue barrier breakdown is performed visually.

Alternatively or additionally, blood-tissue barrier is detected by at least one of, a magnetic resonance imaging (MRI) apparatus, a positron emission tomography (PET) apparatus, a computed tomography (CT) apparatus, and a single positron emission computed tomography (SPECT) apparatus.

Alternatively or additionally, the BBB may be replaced with other biological barriers, including cell membrane, nuclear membrane, skin, mucosal membranes, blood-CSF barrier, and blood-tumor lesion barrier.

Alternatively or additionally, the diffusivity of the exogenous contrast agent is different from that of healthy and pathological tissues.

Alternatively or additionally, the said substance is consisting of a food, a nutrient substance, a drug, a nanoparticle, a compound or its combination that can pass the BBB.

Alternatively or additionally, in some implementations, some molecules that exist naturally in the body or brain, such as N-acetylaspartate, lipid lactate, creatine and glucose, can be used as an endogenous diffusivity contrast agents. The endogenous diffusivity contrast agents can be quantified as an image marker by an internal reference or external reference when pathological change occurs.

Alternatively or additionally, the exogenous substance is administered by injection or orally.

Alternatively or additionally, detecting the transport of an exogenous substance across a blood-brain barrier (BBB) using a diffusion magnetic resonance imaging sequence can be applied for diagnosing a disease, monitoring disease therapy, staging a disease, and assessing therapy effect.

Alternatively or additionally, the present method can be applied for monitoring drug delivery when a drug is used as an exogenous substance.

Alternatively or additionally, characterizing the transport of the exogenous substance across the BBB according to the one or more post-contrast image data can be qualitative or quantitative.

Alternatively or additionally, post contrast agent image data can be implemented to quantify one or more of the target region or lesion size, the target region or lesion volume, signal-to-noise of healthy tissue, diffusivity, anisotropy, but not limited to, permeability of the substances across BBB.

Alternatively or additionally, quantitatively analyzing physio-pathological properties can further comprise comparing the at least one contrast agent or substance concentration curve with a non-enhanced curve.

It should be understood that the above-described subject matter may also be implemented as a computer-controlled apparatus, a computer process, a computing system, or an article of manufacture, such as a computer-readable storage medium.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for detecting a transport of an exogenous substance across a biological barrier using a diffusion magnetic resonance imaging sequence, the method comprising:
administrating the exogenous substance to target regions including human tissues or lesions;
acquiring one or more images of target regions after the administration of the exogenous substance by the diffusion magnetic resonance imaging sequence;
processing the one or more images to identify diffusivity, permeability, or diffusion tensor parameters of the target regions; and
characterizing the transport of the exogenous substance across the biological barrier based on the identified diffusivity, permeability, or diffusion tensor parameters of the target regions.

2. The method of claim 1, further comprising:
acquiring a first image of the target regions before the administration of the exogenous substance by the diffusion magnetic resonance imaging sequence;
acquiring a second image of the target regions after the administration of the exogenous substance by the diffusion magnetic resonance imaging sequence or the diffusion tensor imaging;
identifying changes in diffusivity, permeability, or diffusion tensor parameters of the target regions by comparing the first image and the second image of the target regions; and
characterizing the transport of the exogenous substance across the biological barrier based on the identified changes in diffusivity, permeability, or diffusion tensor parameters of the target regions.

3. The method of claim 2, wherein the changes in diffusivity, permeability, or diffusion tensor parameters of the target regions are identified by subtracting the first image from the second image.

4. The method of claim 1, wherein the biological barrier is one of a blood-brain barrier, cell membrane, nuclear membrane, blood-tissue barrier, skin, mucosal membranes, blood-cerebrospinal fluid barrier, blood-retinal barrier, blood-testis barrier, blood-tumor lesion barrier, and blood brain barrier.

5. The method of claim 1, wherein:
the target regions include healthy tissues and pathological tissues; and
the diffusivity, permeability, or diffusion tensor parameters of healthy tissues is different from the diffusivity, permeability, or diffusion tensor parameters of pathological tissues.

6. The method of claim 1, wherein the exogenous substance consists of a food, a nutrient substance, a drug, a nanoparticle, a compound or combination thereof.

7. The method of any of claims 1-6, wherein the exogenous substance is administered by injection or orally.

8. The method of any of claims 1-7, further comprising at least one of diagnosing a disease, monitoring disease therapy, staging a disease, or assessing therapy effect based on the characterization of the transport of the exogenous substance.

9. The method of any of claims 1-8, further comprising monitoring drug delivery when a drug is used as the exogenous substance.

10. The method of claim 1, wherein characterizing the transport of the exogenous substance across the biological barrier based on the one or more images of the target regions is qualitative or quantitative.

11. The method of claim 1, further comprising:
processing the one or more images to quantify physio-pathological properties including one or more of sizes of the target regions, volumes of the target regions, signal-to-noise of the target regions, contrast-to-noise of the target regions, diffusivity of the target regions, anisotropy of the target regions, and permeability of the substance agent across the biological barrier.

12. The method of claim 11, wherein quantifying physio-pathological properties comprises comparing a concentration curve or the exogenous substance with a non-enhanced curve.

13. The method of claim 1, wherein the diffusion magnetic resonance imaging sequence imaging is one of diffusion weighted imaging, diffusion tensor imaging (DTI), and intra-voxel incoherent motion (IVIM).

14. The method of claim 1, wherein the one or more images are acquired a predetermined time after the administration of the exogenous substance.

15. A system for detecting a transport of an exogenous substance across a biological barrier using a diffusion magnetic resonance imaging sequence, the system comprising:
a coil configured to apply a diffusion magnetic resonance imaging sequence to target regions in a subject;
a receiver configured to acquire one or more images of target regions after the administration of the exogenous substance by the diffusion magnetic resonance imaging sequence; and
a processor configured to:
process the one or more images to identify diffusivity, permeability, or diffusion tensor parameters of the target regions; and
characterize the transport of the exogenous substance across the biological barrier based on the identified diffusivity, permeability, or diffusion tensor parameters of the target regions.

16. The system of claim 15, wherein the processor is further configured to:
receive a first image of the target regions before the administration of the exogenous substance by the diffusion magnetic resonance imaging sequence;
receive a second image of the target regions after the administration of the exogenous substance by the diffusion magnetic resonance imaging sequence;
identify changes in diffusivity, permeability, or diffusion tensor parameters of the target regions by comparing the first image and the second image of the target regions; and
characterize the transport of the exogenous substance across the biological barrier based on the identified changes diffusivity, permeability, or diffusion tensor parameters of the target regions.

17. The system of claim 16, wherein the changes in diffusivity, permeability or diffusion tensor parameters of the target regions are identified by subtracting the first image from the second image.

18. The system of claim 15, wherein the biological barrier is one of a cell membrane, nuclear membrane, blood-tissue barrier, skin, mucosal membranes, blood-cerebrospinal fluid barrier, blood-retinal barrier, blood-testis barrier, blood-tumor lesion barrier, and blood brain barrier.

19. The system of claim 15, wherein the exogenous substance consists of a food, a nutrient substance, a drug, a nanoparticle, a compound or combination thereof.

20. The system of claim 15, wherein the diffusion magnetic resonance imaging sequence imaging is one of diffusion weighted imaging, DTI, and IVIM.

21. The system of claim 15, wherein characterizing the transport of the exogenous substance across the biological barrier based on the one or more images is qualitative or quantitative.

22. The system of claim 15, wherein the one or more images are acquired a predetermined time after the administration of the exogenous substance.

23. The system of claim 15, wherein the processor is further configured to perform at least one of diagnosing a disease, monitoring disease therapy, staging a disease, assessing therapy effect, and monitoring drug delivery based on the characterization of the transport of the exogenous substance.
